(19) United States
(12) Patent Application Publication
(10) Pub. No.: US 2002/0188301 A1
(43) Pub. Date: Dec. 12, 2002

(54) Tissue anchor insertion system

(76) Inventors: Mark Douglas Dallara, Tampa, FL (US); Drew Powell Amery, Clearwater, FL (US)

Correspondence Address: GENE WARZECHA, LINVATEC CORPORATION, 11311 CONCEPT BOULEVARD, LARGO, FL, 33773

(21) Appl. No.: 09/878,610

(22) Filed: Jun. 11, 2001

(51) Int. Cl. A61B 17/60

(52) U.S. Cl. 606/104

(57) ABSTRACT

A system for inserting a tissue anchor during endoscopic or other surgical procedures. The system incorporates an insertion instrument for inserting into a surgical site of implantation an elongated, tissue anchor having, for example, a plurality of barbs outwardly extending from its body and a transverse head situated at its proximal end. The instrument is provided with an elongated needle received in the cannulation of the anchor, and an elongated pusher for pushing the anchor into tissue. The needle and pusher are fixedly attached to a handle and are situated in the lumen of a slidable, distally biased sheath or cannula for maintaining the tissue anchor, needle and pusher in alignment while the handle is pushed. The anchor may be inserted by a single-handed operation of the instrument through a simple pushing motion which causes the sheath to retract simultaneously with advancement of the handle to allow the anchor to be pushed into tissue.

### BACKGROUND OF THE INVENTION

1. Field of the Invention

The invention relates to implant devices and instruments used to repair body tissue. In particular, the invention relates to implant devices, instruments and methods for repairing body tissue during endoscopic surgical procedures. Still more particularly, the invention relates to implant devices, instruments and methods for repairing meniscal tissue during arthroscopic surgery of the knee.

### DESCRIPTION OF THE PRIOR ART

Implant devices for repairing body tissue are known in the prior art. While such devices may be classified into several categories, the present invention is related to instruments and methods for inserting into a site of implantation elongated devices having transversely extending barbs or projections which assist in retaining the implant in place within a tissue defect (e.g. a tear) to hold body tissue in close approximation for healing or other reasons. It will be understood that the invention can also be used to drive devices such as headed tacks or suture anchors with eyelets or other suture retaining members, where such devices are used to approximate tissue to an underlying base such as bone or other tissue (even soft tissue).

One such known device is described in U.S. Pat. No. 4,873,976 (Schrieber). This device comprises a solid elongated shaft having a plurality of transversely extending projections, a pointed tip and a transverse circular head at its proximal end. The Schrieber device is inserted at a surgical site of implantation by being pushed through an elongated hollow tube (cannula) which is held next to, but does not itself penetrate the site of implantation. In one commercial embodiment of this device, the distal end of the cannula is placed against tissue at the chosen site of implantation and a needle is pushed through the cannula from its proximal end (outside the body) to its distal end and into the tissue a predetermined amount. The needle is then removed and the implant device is inserted into the proximal end of the cannula. The implant device is then pushed by an obturator entirely through the cannula and into the tissue. The proximal end of the obturator may need to be tapped to drive the implant into the tissue.

Other similar devices are disclosed in U.S. Pat. Nos. 4,884,572; 4,895,148; 4,924,865; and 4,976,715 all issued to Bays et al. The devices disclosed in these Bays et al. patents primarily differ from the Schrieber device in that they are cannulated. The Bays et al. patents are assigned to the assignee hereof and, along with the Schrieber patent, are incorporated by reference herein. The Bays et al. device is inserted at a surgical site of implantation with an applicator having a needle passing through an axial bore of the applicator and through an axial bore of the implant. The implant is held at the tip of the applicator and inserted into the site of implantation directly through a portal or through an insertion cannula. The needle protrudes distally from the implant and both the needle and implant are pushed into the tissue while so assembled. The needle is then disassembled from the applicator and removed. In the Bays et al. patents the needle and the implant are not protected by any surrounding sheath and are simply pushed distally when a user pushes the applicator distally.

All of the above described elongated devices are arrow-like and are designed to be inserted or pushed into tissue to be repaired. The devices are sometimes referred to as “tissue anchors” because they hold tissue together during healing. While these devices are intended to be used during arthroscopic or, more generally, endoscopic procedures, that very fact makes the insertion sometimes difficult. As described above, it is known to use elongated cannulas to guide the implants into position and smaller push rods to push them in. Insertion devices and methods used with the Schrieber type non-cannulated device require the implant to be pushed through a cannula with an elongated pusher sized to be slidingly received within the cannula. Insertion devices and methods used with cannulated devices such as those disclosed in the Bays et al. patents require the implant device to be secured to the distal tip of a holding device and pushed into place, with or without the use of a guiding cannula.

An improved cannulated implant and insertion system is described in pending U.S. Pat. No. 6,146,387 (Trott et al.) entitled Cannulated Tissue Anchor System, assigned to the assignee hereof and incorporated by reference herein. The insertion system shown in this patent comprises a housing, an elongated tubular shaft extending distally from the housing, the shaft having an axially aligned bore therethrough and an elongated needle adapted to be slidably received within the bore of the shaft. The shaft is adapted to receive a cannulated tissue anchor while the needle is adapted to be received in the bore of the anchor. A trigger means is provided on the housing for moving the distal end of the needle between a first, retracted position, in which the needle is maintained within the shaft bore, and a second, extended position, in which the needle is extended distally, beyond the shaft bore. A push rod for pushing the anchor out of the device is adapted to be slidably received within the shaft bore and moved between a first, retracted position, in which the distal end of the push rod is maintained within the shaft bore, and a second, extended position, in which the distal end of the push rod is adjacent or slightly beyond the distal end of the shaft.

Another improved insertion system is described in U.S. Pat. No. 6,074,395 (Trott et al.) entitled Cannulated Tissue Anchor Insertion System, assigned to the assignee hereof and incorporated by reference herein. This patent shows a system similar to that of the aforementioned U.S. Pat. No. 6,146,387, but wherein the movement of the needle and push rod are controlled by sequential pulls of a single trigger.

It is always desirable to simplify the insertion process for push-in, arrow-like implant devices. Accordingly, it is an object of this invention to develop a tissue repair system incorporating a cannulated push-in implant or tissue anchor device, preferably bioabsorbable, and a simplified insertion apparatus, preferably operable by one hand.

It is therefore also an object of this invention to provide a tissue anchor inserting device and method for guiding and inserting a cannulated tissue anchor into position at a surgical site.

It is another object of this invention to provide an elongated inserting device for receiving therein a cannulated tissue anchor, preferably at its distal end.

It is still another object of this invention to provide an elongated inserting device suitable for endoscopic procedures and capable of being operated from its proximal end.

It is an additional object of this invention to provide a disposable insertion instrument for use with cannulated tissue anchors.

It is also an object of this invention to provide an insertion instrument suitable for use with non-cannulated tissue anchors.

It is another object of this invention to provide an insertion instrument suitable for inserting into soft tissue or bone any implant such as suture anchors and headed tacks.

### SUMMARY OF THE INVENTION

These and other objects are accomplished by the preferred embodiment of the system disclosed herein which comprises a surgical instrument for inserting a cannulated surgical implant into a surgical site. The instrument comprises a handle having an elongated needle and an elongated pusher fixedly connected to the handle. The needle is adapted to receive a cannulated tissue anchor and the anchor, needle and pusher are encased within a slidable, retractable sheath or cannula. The sheath maintains the anchor, needle and pusher in alignment to provide column support to the anchor to allow the anchor to be pushed into tissue in a direction in alignment with the anchor axis. Distal movement of the sheath is resisted by the tissue as the anchor is pushed distally, thereby effectively causing the sheath to move proximally relative to the anchor, needle and pusher. The needle and pusher are withdrawn once the anchor is set at the desired depth. The retractable sheath protects inadvertent damage to tissue during insertion and withdrawal.

In a preferred embodiment, the instrument comprises a surgical instrument for inserting a cannulated tissue anchor implant into tissue at a surgical site, the implant having a distal end, a proximal end and an axially aligned bore therethrough. The instrument comprises a handle having a distal end and a proximal end, an elongated push rod extending distally from the distal end of the handle, and a needle extending distally from the distal end of the push rod. The needle is adapted to be received within the bore of the implant and the push rod has a distal end adapted to push the cannulated implant distally. An elongated tubular sheath extends distally from the distal end of the handle. The sheath has an axially aligned lumen within which the push rod and the needle are received. The sheath has a predetermined length, sufficient to receive the needle within the lumen and a predetermined diameter, sufficient to receive the implant in the lumen. A spring means is situated between the handle and the sheath for biasing the sheath distally. The spring means is adapted to be overcome by a predetermined amount of proximally directed force applied to the distal end of the sheath to thereby expose the needle as the sheath is moved proximally.

The invention also resides in the method of using the aforementioned instrument to place a cannulated surgical implant at a surgical site with an instrument suitable for single-handed use. The method comprises the steps of providing a cannulated tissue anchor and a tissue anchor inserter as described above. The method further comprises the steps of positioning the implant on the needle and within the distal end of the elongated sheath and positioning the distal end of the sheath at a selected site of implantation. As the end of the sheath is pushed against tissue, its distal motion is prevented while the distal motion of the anchor, needle and pusher continues, thus simultaneously moving the needle, the pusher and the implant into the surgical site. Such motion causes the slidable sheath to be pushed proximally relative to the needle, pusher and implant. The needle and pusher are withdrawn from the site of implantation, allowing the sheath to extend distally to cover the needle.

### BRIEF DESCRIPTION OF THE DRAWINGS

FIG. 1 is a side elevational view of a prior art cannulated surgical implant in the form of a tissue anchor suitable for use with an insertion instrument constructed in accordance with the principles of this invention.

FIG. 2 is a left end view of FIG. 1.

FIG. 3 is a side elevational view of a cannulated implant insertion instrument constructed in accordance with the principles of this invention.

FIG. 4 is a cross-sectional view of the instrument of FIG. 3 showing its internal components and showing the tissue anchor of FIG. 1 situated at its distal end.

FIG. 5 is a view of FIG. 4 showing the instrument during a portion of the process of implanting the tissue anchor.

FIG. 6 is an exploded view of the instrument of FIG. 3.

FIGS. 7 and 8 are views of some components of the instrument of FIG. 6 prior to their assembly.

FIGS. 9 through 11 are views of another component of the instrument of FIG. 6.

FIGS. 12 through 16 are sketches of various steps in the method of using the instrument of FIG. 3.

### DESCRIPTION OF THE PREFERRED EMBODIMENT

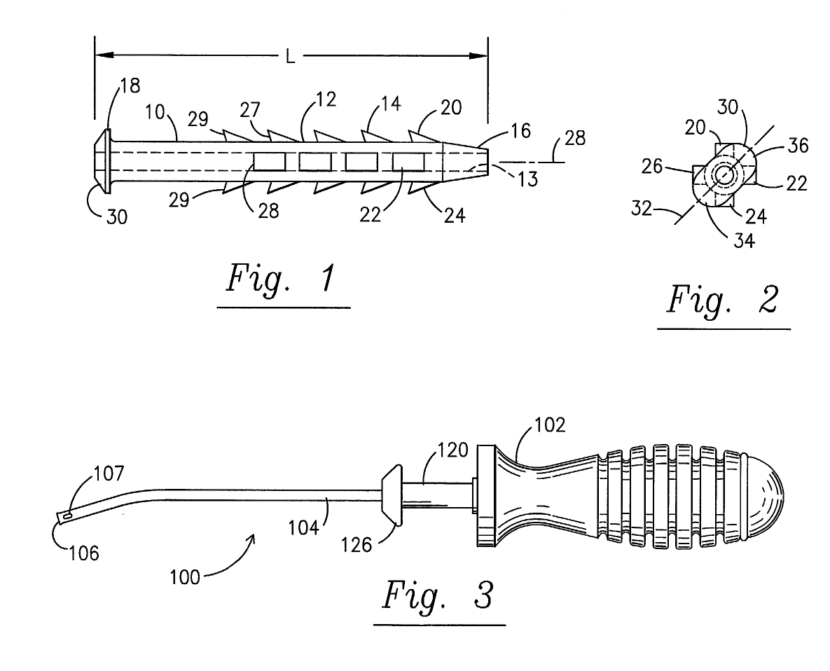

Referring now to FIGS. 1 and 2 there is shown a cannulated tissue anchor 10 described in the aforementioned Trott et al. patents. This anchor is described as an example of the type of anchor intended to be used with the insertion system shown in FIGS. 3 through 11. Those skilled in the art will understand that the insertion system can also be used with other types of implants such as non-cannulated implants, suture anchors and headed tacks.

Anchor 10 comprises an elongated shaft 12 having an axial bore 13 and a plurality of barbs 14 situated on its external surface and extending between a distal end 16 and a proximal end 18. The barbs are arranged in four linear rows 20, 22, 24 and 26 with rows 20 and 24 having an equal number of barbs in each row and rows 22 and 26 having a lesser number of barbs in each row. The barbs in adjacent rows are longitudinally staggered to enable the tissue anchor to resist rotation about its axis 28. The anchor may be made in various lengths and diameters with various numbers of barbs and with various lengths of smooth, barb-free shafts between the proximal most barbs 29 and proximal end 18. In the preferred embodiment, all rows have three barbs each if the anchor length L is 10 mm. If the anchor length L is 13 mm or 16 mm, rows 20 and 24 each have five barbs and rows 22 and 26 each have four barbs (as shown in FIG. 1). The distal-most barbs in all cases are situated at the same distance from distal end 16.

Anchor 10 further comprises a head 30 at its proximal end 18. In the preferred embodiment, head 30 is a generally flat, oval structure having a major axis 32 which is angled relative to the plane of rows 20 and 24 as best seen in FIG. 2. This intentional misalignment of the axis of head 32 enables it to abut tissue in the areas adjacent to the distally facing sides of portions 34 and 36 of the head. It will be understood that as the barb rows 20, 22, 24 and 26 are pushed into tissue to be treated at the surgical site, the tissue is necessarily pushed aside or slightly deformed in the areas adjacent the barbs and along the lines of the barbs. If the head axis 32 were to be aligned in the plane of two diametrically opposed rows of barbs, for example, the head may have a tendency to migrate distally along the tissue defects created by the barb rows. The intentional misalignment of the axis of the head prevents the distal advancement of the barb because the head lies adjacent “virgin” tissue which is not subject to deformation by the barb rows. Thus, it will be understood that the particular shape and orientation of head 30 enables the profile of the head to be minimized while also minimizing the possible migration of the tissue anchor at or from the surgical site. This beneficial orientation of the major axis of the head would also apply to tissue anchors in which the barbs might be arranged in helical rows.

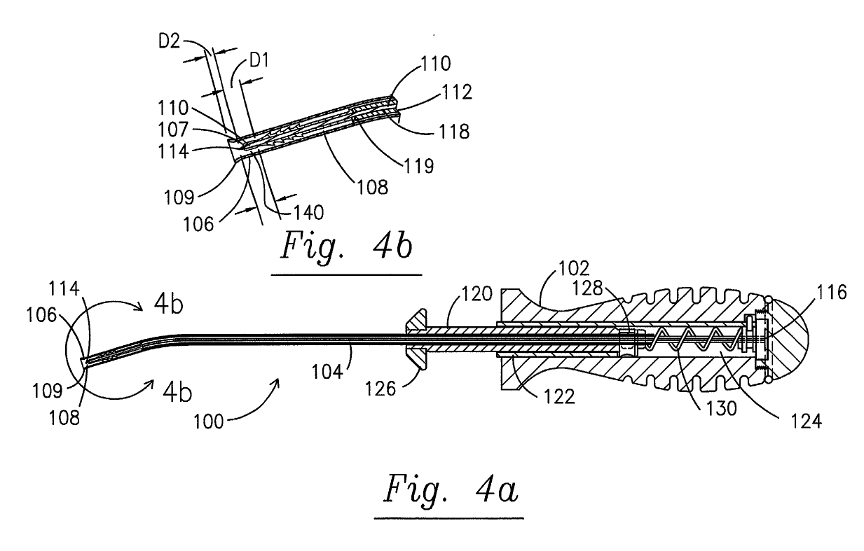

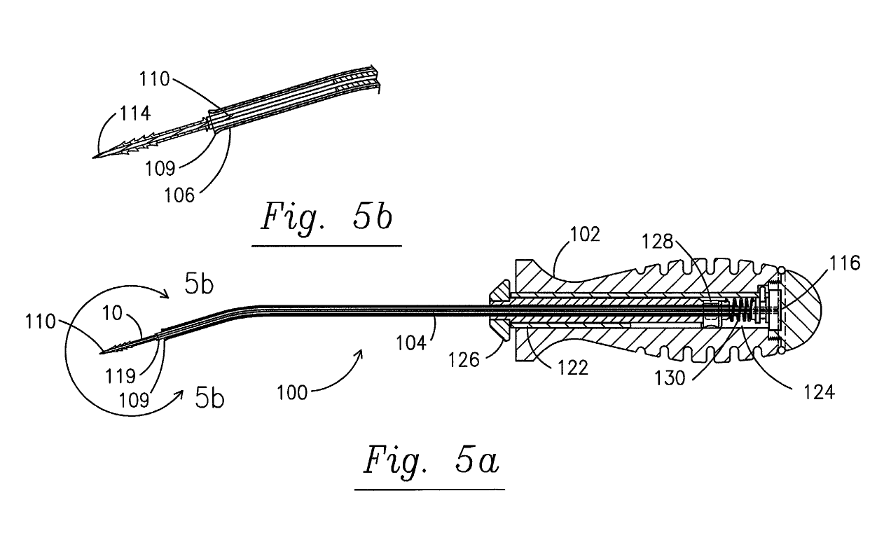

Referring now to FIGS. 3 through 11, there is described a preferred tissue anchor inserter system for inserting a cannulated tissue anchor (such as anchor 10). The system comprises an instrument or inserter 100, preferably made to be disposable, and having a handle 102 at its proximal end and a slidable sheath or cannula 104 extending distally from the distal end of the handle. Instrument 100 may be produced and shipped with a tissue anchor pre-loaded as shown in FIG. 4. However, it may be preferable to ship the instrument without the anchor. As best seen in FIGS. 4 and 5, cannula 104 has an open distal end 106 and an axially aligned lumen 108 within which are situated an elongated needle 110 and pusher rod or pusher 112. Cannula end 106 is provided with a fenestration or window 107 to enable a user to see the position of the tissue anchor and has a distally facing circular rim 109 which may be flared slightly or rounded to minimize forces exerted on tissue as will be understood below. Rim 109 may be provided with friction enhancing features (e.g. points or roughened surface) to enhance the contact with the tissue. This is more significant if the inserter is adapted for use with a non-cannulated implant as will be discussed below. Needle 110 has a pointed distal end 114 and a proximal end 116 fixedly connected to the proximal end of handle 102. In the preferred embodiment, needle 110 may be made of a stainless steel or a memory alloy such as nitinol and has a diameter of 0.025 inches (0.635 mm) to fit in anchor bore 13 which has a diameter of 0.026 inches (0.660 mm).

Elongated pusher 112 is interposed between needle 110 and the wall of the cannula lumen 108 and is axially cannulated with bore 118 to receive the needle. In the preferred embodiment pusher 112 is also fixedly attached to the proximal end of handle 102. Alternatively, pusher 112 and needle 110 may be made as an integral member having a solid, non-cannulated pusher body extending proximally from the annular, anchor-head contacting surface 119, and a solid needle portion extending distally therefrom. In the preferred embodiment, pusher 112 may be made of a suitable metal such as stainless steel or a suitable plastic having sufficient column strength to push the implant. Pusher 112 may, for example, be a spring coil in which each turn of the coil abuts the adjacent turns, thus providing longitudinal strength even when the spring coil is curved. Lumen 108 has a diameter sufficient to receive anchor 10 and pusher 112. While it is preferred that the distal end of the pusher be circular and have a diameter equal to the major diameter of the anchor, this may not be necessary in all situations. Furthermore, while the distal end of the pusher contacting the anchor may be one size, the remainder of the pusher, extending proximally from the anchor, may be larger or smaller in diameter (and may also be non-circular).

Cannula 104 is slidable because it is secured to a slide or holder 120 interposed between the handle and the cannula, the holder being slidably situated within a guide bearing 122 secured within an internal bore 124 of handle 104. It will be understood that the functions of the guide bearing may be incorporated into the handle body to obviate the need for a separate part. Holder 120 has a circular control knob/stop 126 at its distal end and is secured to the cannula at its proximal end 128 so as to be slidable therewith. In the preferred embodiment, holder 120, guide bearing 122 and bore 124 each generally have a circular cross-section although this is not essential so long as they are shaped so as to be slidable relative to each other. Compression spring 130 is situated between the proximal end of holder 120 and the proximal end of bore 124 in order to bias holder 120 and cannula 104 distally. The spring must be strong enough to bias the cannula distally, but must allow the cannula to slide proximally relative to needle 110 as instrument 100 is pushed distally into tissue. Alternatively, a different spring arrangement could be used. For example, the spring could be anchored distally of its point of attachment to holder 120 to provide a distally directed bias. Also alternatively, the cannula body could include a helically or transversely slotted section to provide a spring action. It will be understood that spring 130 is not essential to the operation of the invention although it is desirable in the preferred embodiment. If there is no spring, the surgeon may manipulate the cannula distally by simply pushing the cannula manually.

If instrument 100 is shipped without a tissue anchor, a tissue anchor must be loaded onto the needle of the instrument prior to use. The distal end of the tissue anchor/inserter assembly in this loaded condition is shown in greater detail in the enlarged portion of FIG. 4. It will be noted that in this loaded configuration, with cannula 106 fully extended distally by spring 130, the anchor resides on needle 110 and within an annular chamber 140 at the open distal end of inserter 100, the proximal side of head 30 of the tissue anchor abuts the annular distally facing surface of pusher rod 112 and the distal tip 114 of needle 110 projects a predetermined distance Dl beyond the tip of the anchor. The needle tip is at this stage situated proximally of the rim 109 by a predetermined distance D2. The diameter of chamber 140 is only slightly larger than the diameter of the anchor head 130 to provide column support. As will be understood below, and as shown in FIG. 5, this configuration of anchor, needle and pusher is maintained while the cannula 104 slides proximally due to its interaction with tissue (not shown) against the cannula end rim 109. When fully retracted, the cannula's control knob/stop 126 will abut the distal end of the guide bearing while end 109 is retracted (as shown in FIG. 5) to enable the pusher to countersink the anchor below the surface of the tissue contacting rim 109. That is, the anchor is pushed in sufficiently far that when pressure is released the head of the anchor will create a “dimpled” effect on the surface of the tissue.

Instrument 100 is shown with its cannula 104 having a curved distal end, although it will be understood that straight or other various simple or compound curves could be formed in the distal end to enable the implant to be endoscopically or otherwise delivered to a variety of surgical sites. An example of possible curves is shown in the aforementioned U.S. Pat. No. 6,146,387.

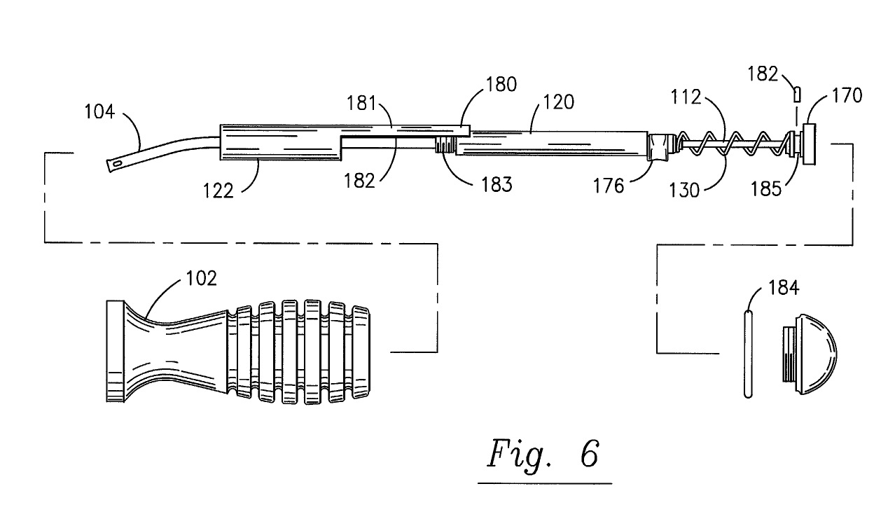

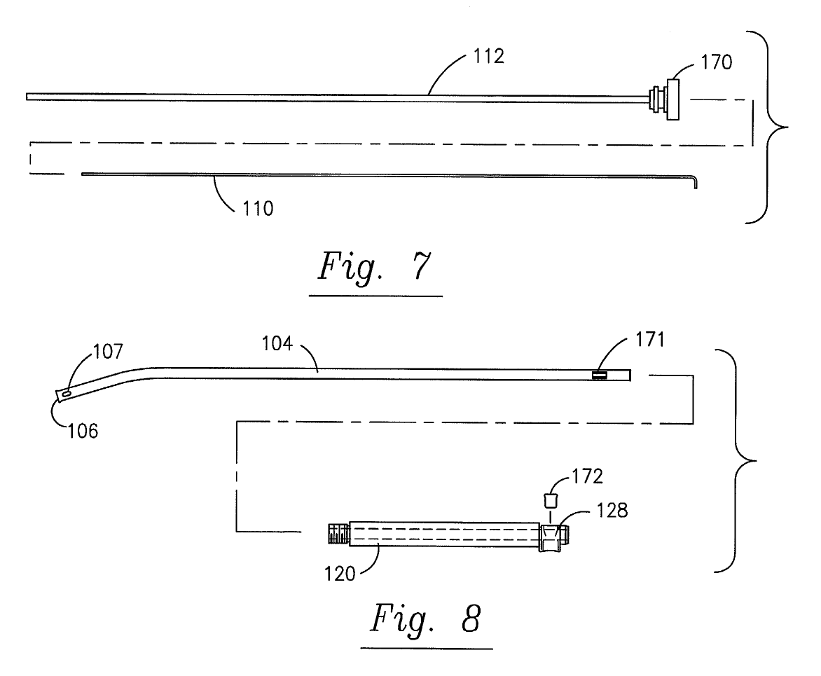

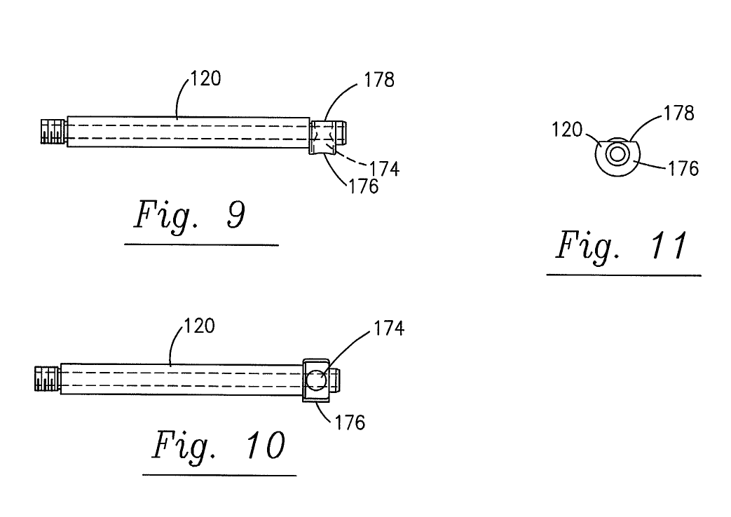

The assembly of the components of the preferred embodiment of instrument 100 is shown in FIGS. 6, 7 and 8. In FIG. 6, the components identified above fit together as shown while additional components shown in FIGS. 6, 7 and 8 facilitate the assembly. Thus, as shown in FIG. 7, cannulated locator disc 170 is threaded or otherwise secured to the proximal end of pusher 112 and needle 110, having a bent proximal end is inserted into the bore of the pusher so the bent end abuts the locator disc. As shown in FIGS. 8-11, the proximal end of cannula 104 is inserted into the axial bore of holder 120, best seen in elevational cross-section, plan and end views, respectively, in FIGS. 9-11. To secure holder 120 to cannula 104, the proximal end of the cannula is slotted or otherwise shaped at 171 (in the preferred embodiment with a circular bore) to receive a transverse pin 172 inserted through a transverse bore 174 in the proximal end of the holder. The proximal end of the holder is further provided with a circular boss 176 having a flat 178 on one side. The circular part of the boss slidably mates with the circular wall of bore 124 while flat 178 enables slidable, non-rotating motion between holder 120 and guide bearing 122. For this purpose guide bearing 122 is provided with a proximally extending extension 180 having an arcuate radially outer surface 181 and a pair of flat radially inner surfaces 182, separated by an arcuate section conforming to the outer surface of holder 120. The abutment of the distal side of bore 124 with the proximal end of guide bearing 122 limits distal motion of cannula 104. The distal end 183 of holder 120 is threaded to receive control knob 126.

Referring now to FIG. 6, the pusher/needle assembly of FIG. 7 is inserted through spring 130 into the proximal end of the holder/cannula assembly of FIG. 8. Cannula 104 is then inserted through the axial bore of guide bearing 122 into the body of handle 102. The proximal end of handle 102 is adapted to receive locator disc 170 so as to limit its motion in a distal direction (in the preferred embodiment the diameter of bore 124 is less than the diameter of the locator disc). Locator disc 170 is secured to extension 180 by roll pin 182 extending through a hole (not shown) in the extension and set into groove 185. O-ring 184 and handle cap 186 facilitate fixed attachment of the pusher and needle to the handle as well as securing the various components to the handle body.

While the various components may be made of any compatible materials which can perform the above-described functions, in the preferred embodiment the handle, holder and pusher are made of various polymeric materials while the cannula and guide bearing are made of suitable grades of stainless steel, as will be understood by those skilled in this art. The relative lengths of the components may be changed depending upon the length of the tissue anchor and the desired insertion depth. The cannula should be long enough to laterally enclose the needle and pusher.

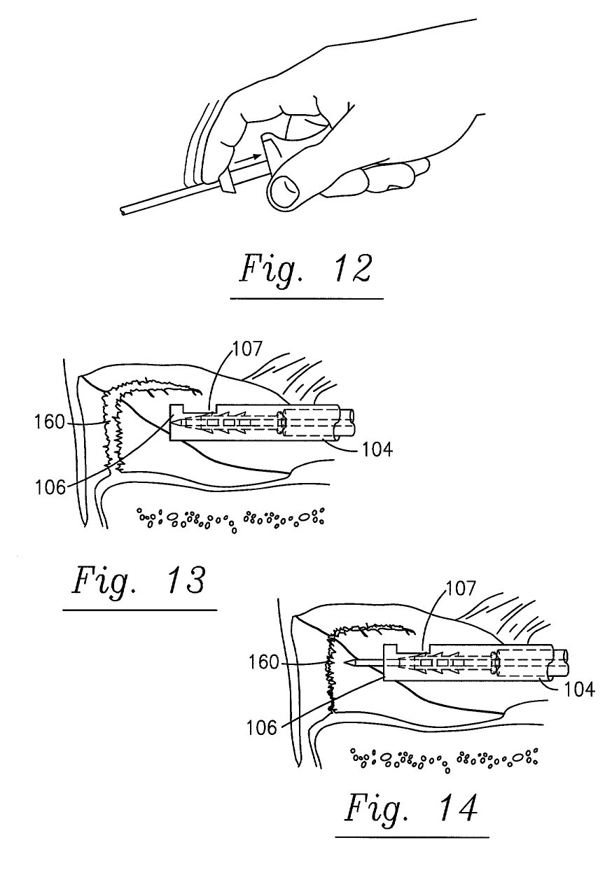

The explanation of the operation of inserter 100 will be best understood by reference to the method shown in FIGS. 12 through 16.

Because the insertion instrument is made in specific sizes, the user must select the appropriate implant size and match it with the corresponding inserter size based on location and size of the tear. The first step is to load the tissue anchor on the distal end of needle 110. Retracting the cannula 104 as shown in FIG. 12 will expose the needle and so that a tissue anchor may be placed on the needle. The cannula may then be released so the pressure of spring 130 will cause it to move distally to thereby cover and secure the tissue anchor. The assembled anchor/inserter is now ready for insertion into the patient and advancement of the distal end 106 to the surgical site to repair, for example, a tissue tear 160 as shown in FIG. 13. (While repair of meniscus is shown here, it will be understood that other tissue may also be repaired.) The cannula is then retracted approximately 2 mm by pulling back on the control knob to expose the needle tip as shown in FIG. 14. This enables the user to reduce the tear with the tip of the needle. Once the tear is reduced, the distal control knob 126 is released. It will be understood that the anchor is held in place within chamber 140 by frictional engagement with the needle and/or the inner wall of the cannula. The user may prevent inadvertent proximal motion of cannula 104 during instrument insertion, and possible premature release of the anchor, by simply keeping control knob 126 fixed in place with an index finger.

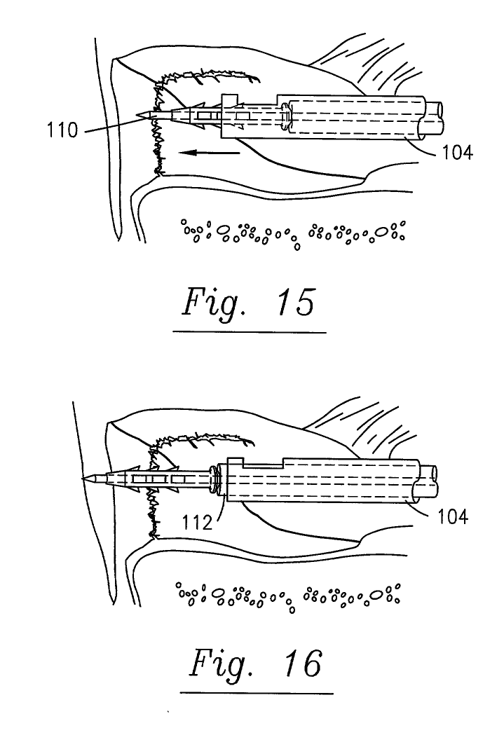

After the cannula tip 109 is placed against the meniscus surface, the handle 102 is pushed forward without holding control knob 126 to thereby push the needle and pusher, and consequently expose the needle and drive the implant 10 across the tear as shown in FIG. 15. In order to maintain support for the implant and needle and optimize column strength, the spring 130 will keep the cannula tip 109 against the meniscus surface during insertion, thus assuring axial alignment of the implant and needle. Flaring or rounding tip 109 may minimize any tendency for the cannula to penetrate the tissue. Tip 109 may be rounded, blunted, flanged or otherwise provided with a design to decrease the force per unit area which the tip applies to the tissue.

As shown in FIG. 16, the pusher 112 may be made to extend slightly beyond the distal tip 109 in order to “countersink” the head of the tissue anchor into the tissue.

While the method described above can be initiated by the manual loading of a single cannulated tissue anchor assembly onto the insertion device, a plurality of tissue anchors may alternatively be held in a modified device (not shown) which would sequentially load an anchor into position at the distal end of the anchor assembly tube so that a plurality of anchors could be applied without having to remove the instrument to reload another single tissue anchor assembly.

While the handle 102 is shown in line with the cannula 104, it will be understood that other types of handles may be used. For example, a pistol grip handle (not shown) would also be suitable.

While the aforementioned embodiment of the invention has been described as incorporating a needle, it will be understood that the invention can also be inserted in a device having only a sheath and a pusher, and suitable for replacing non-cannulated implants. That is, the needle need not be used if the implant is shaped so as to be implantable alone, without the aid of a needle.

It will be understood by those skilled in the art that numerous improvements and modifications may be made to the preferred embodiment of the invention disclosed herein without departing from the spirit and scope thereof.

What is claimed is:

1. A surgical instrument for inserting a tissue anchor implant into tissue at a surgical site, said implant being cannulated and having a distal end, a proximal end and an axially aligned bore therethrough, said instrument comprising:
a handle having a distal end and a proximal end;
an elongated push rod extending distally from said distal end of said handle, said push rod having a distal end adapted to push the cannulated implant distally;
an elongated needle extending distally from said distal end of said push rod, said needle adapted to be received within the bore of the implant;
an elongated tubular sheath extending distally from said distal end of said handle, said sheath having an axially aligned lumen within which said push rod and said needle are received, said sheath having a predetermined length which is sufficient to receive said needle within said lumen and said sheath having a predetermined diameter which is sufficient to receive said implant in said lumen;
a spring means situated between said handle and said sheath for biasing said sheath distally relative to said handle, said spring means adapted to be overcome by a predetermined amount of proximally directed force applied to the distal end of said sheath to thereby expose said needle as said sheath moves proximally.

2. A surgical instrument according to claim 1 wherein said distal end of said sheath is provided with friction enhancing means to facilitate contact with tissue.

3. A surgical instrument according to claim 1 wherein said spring means comprises a helical slot in the wall of said cannula.

4. A surgical instrument according to claim 1 wherein said handle has a hollow interior and said sheath has a proximal end situated in said handle, said proximal end of said sheath further comprising a stop means to limit distal longitudinal motion of said sheath.

5. A surgical instrument according to claim 1 wherein said elongated pusher comprises a cylindrical tube having an axially aligned bore for receiving said elongated needle therethrough.

6. A surgical instrument according to claim 1 wherein said elongated push rod has a proximal end and is fixedly attached at its proximal end to said handle.

7. A surgical instrument according to claim 5 wherein said elongated needle has a proximal end and is fixedly attached at its proximal end to said handle.

8. A surgical instrument for inserting a cannulated surgical implant into a surgical site, the instrument comprising:
an elongated needle for slidably receiving a cannulated implant thereon, said needle having a proximal end and a distal end;
an elongated push rod axially aligned with said needle for pushing said implant distally with said needle, said push rod having a proximal end and a distal end, said distal end of said push rod fixedly situated a predetermined distance proximally from said distal end of said needle;
an elongated tubular sheath for laterally enclosing said needle and said push rod, said sheath having an open distal end for enabling said needle to pass therethrough; and
spring means for biasing said sheath distally and enabling it to move proximally relative to said needle and push rod in response to proximally directed force on said open distal end.

9. A surgical instrument for inserting a tissue anchor implant into tissue at a surgical site, said implant being cannulated and having a distal end, a proximal end and an axially aligned bore therethrough, said instrument comprising:
a handle having a distal end and a proximal end;
an elongated push rod extending distally from said distal end of said handle, said push rod having a distal end adapted to push the cannulated implant distally;
an elongated needle extending distally from said distal end of said push rod, said needle adapted to be received within the bore of the implant;
an elongated tubular sheath extending distally from said distal end of said handle, said sheath having an axially aligned lumen within which said push rod and said needle are received, said sheath having a predetermined length which is sufficient to receive said needle within said lumen and said sheath having a predetermined diameter which is sufficient to receive said implant in said lumen;
means for biasing said sheath distally relative to said handle, said means adapted to be overcome by a predetermined amount of proximally directed force applied to the distal end of said sheath to thereby expose said needle as said sheath moves proximally.

10. A surgical instrument according to claim 9 wherein said means for biasing said sheath is a spring.

11. A surgical instrument according to claim 9 wherein said means for biasing said sheath is manually activated and dependent on force applied by a user of the instrument.

12. A surgical instrument for inserting a tissue anchor implant into tissue at a surgical site, said implant being non-cannulated and having a distal end and a proximal end, said instrument comprising:
a handle having a distal end and a proximal end;
an elongated push rod extending distally from said distal end of said handle, said push rod having a distal end adapted to push the non-cannulated implant distally;
an elongated tubular sheath extending distally from said distal end of said handle, said sheath having an axially aligned lumen within which said push rod is received, said sheath having a predetermined length which is sufficient to receive said push rod within said lumen, with said implant situated in alignment therewith at the distal end of said push rod, and said sheath having a predetermined diameter which is sufficient to receive said implant in said lumen;
means for biasing said sheath distally relative to said handle, said means adapted to be overcome by a predetermined amount of proximally directed force applied to the distal end of said sheath to thereby expose said needle as said sheath moves proximally.

13. A surgical instrument according to claim 12 wherein said means for biasing said sheath is a spring.

14. A surgical instrument according to claim 12 wherein said means for biasing said sheath is manually activated and dependent on force applied by a user of the instrument.

15. A method for implanting a cannulated surgical implant into tissue at a site of implantation comprising the steps of:
providing a cannulated surgical implant having a bore therethrough;
providing a surgical implant inserting instrument comprising an elongated needle for slidably receiving a cannulated implant thereon, said needle having a proximal end and a distal end;
an elongated push rod axially aligned with said needle for pushing said implant distally with said needle, said push rod having a proximal end and a distal end, said distal end of said push rod fixedly situated a predetermined distance proximally from said distal end of said needle;
loading said implant on said needle, with said needle situated within said bore and said implant situated within the open distal end of said tubular sheath;
positioning said open distal end of said tubular sheath at a selected site of implantation;
pushing said instrument distally to thereby simultaneously move said needle, said push rod and said implant into the tissue at the site of implantation, such motion causing said tubular sheath to contact said tissue and to be pushed by said tissue proximally relative to said needle, said push rod and said implant; and
withdrawing said instrument to thereby withdraw said needle from the site of implantation.

16. A method according to claim 15 wherein said step of loading further comprises:
retracting said tubular sheath to expose said needle; and
after inserting said needle through said bore of said implant, releasing said sheath so it is moved distally by said spring means.
